(19) United States
(12) Patent Application Publication
(10) Pub. No.: US 2010/0041968 A1
(43) Pub. Date: Feb. 18, 2010

(54) IMAGE CAPTURE IN COMBINATION WITH VITAL SIGNS BEDSIDE MONITOR

(75) Inventors: Kathleen R. Meschisen, Acton, MA (US); Martin K. Mason, Andover, MA (US); Joseph R. Fallon, Boxford, MA (US); Richard James Conrad, Hamilton, MA (US)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N.V., Eindhoven (NL)

(21) Appl. No.: 12/595,266

(22) PCT Filed: Mar. 26, 2008
(86) PCT No.: PCT/IB2008/051134

§ 371 Date: Oct. 9, 2009

Related U.S. Application Data
(60) Provisional application No. 60/911,293, filed on Apr. 12, 2007.

(51) Int. Cl. A61B 5/00; A61B 6/00

(52) U.S. Cl. 600/301; 600/476

(57) ABSTRACT

When monitoring a patient (32), objective data is readily collected using a vital signs monitor (VSM) (12) coupled to the patient (32) via a plurality of sensors (20). Subjective data, such as the appearance of a rash, wound or dressing, facial pallor or flush, facial expression indicative of pain, and the like, are additionally captured using an imager (22) coupled to the VSM (12). The imager (22) can be a two-dimensional barcode reader that captures a digital image of the patient (32) or portion thereof and relays the image data to the VSM (12). Images and patient vital sign data are then stored to an electronic medical record (14) and presented to a user or physician. Additionally, a standard reference color plate (30) with an optional barcode (34) can be placed on or near the patient (32), and the patient (32) and SCP (30) can be imaged. The imaged SCP (30) is then employed as a reference to perform color correction to permit a reviewing physician to evaluate the image for diagnosis.

### DESCRIPTION

The present application finds particular application in patient healthcare systems, particularly involving vital signs monitors. However, it will be appreciated that the described technique may also find application in other types of monitors or devices, other monitoring scenarios, or other device configuration techniques.

Current vital signs/bedside monitors record the basic measurement data such as SpO₂, etCO₂, blood pressure, temperature, ECG, and heart rate. The clinician will then typically need to make a manual entry (written or typed) into the patient record to describe specific subjective patient conditions (wound healing, pallor, etc). Manual description of a patient condition consumes clinician time to accurately describe the situation. It is typically done subsequently to the time at which vital signs may have been recorded. The entry relies on the clinician's recollection of the condition. In addition, the attending physician will spend time reading the manual description during rounds. Moreover, the physician must rely on the clinician's subjective interpretation of patient condition, and the interpretation may vary from day to day and/or between clinicians.

There is an unmet need in the art for systems and methods that overcome the above-referenced problems and others.

In accordance with one aspect, a system for concurrently capturing vital sign data and an image of a patient includes a vital signs monitor that receives patient parameter data and image data, and an imager, coupled to the VSM, that captures an image of a patient and transmits image data to the VSM. The system additionally includes one or more sensors that monitor at least one patient vital sign and transmit patient parameter data describing the at least one vital sign to the VSM.

In accordance with another aspect, a method of concurrently capturing vital sign information and an image of a patient includes connecting one or more sensors to a patient and to a vital signs monitor (VSM), receiving patient parameter data descriptive of the patient's vital signs at the VSM, and capturing an image of the patient. The method further includes receiving the captured image at the VSM, storing the captured image and the patient parameter data to an electronic medical record (EMR), and displaying the patient parameter data and the captured image.

In accordance with another aspect, a system that facilitates configuring multiple patient monitoring devices includes means for monitoring patient vital signs, means for capturing an image of a monitored patient, and means for storing patient vital sign information and a captured image of the patient as an electronic medical record. The system further includes means for providing a standard reference for color correction of the captured image.

One advantage resides in reducing subjectivity in clinician interpretation of visual patient condition indicators

Another advantage resides in reducing generation and review time associated with recording subjective patient condition information.

Yet another advantage resides in providing a standard reference color plate and color-calibrating patient images to compensate for shadows, poor lighting, and other undesirable influences at the image site.

Still further advantages of the subject innovation will be appreciated by those of ordinary skill in the art upon reading and understand the following detailed description.

The innovation may take form in various components and arrangements of components, and in various steps and arrangements of steps. The drawings are only for purposes of illustrating various aspects and are not to be construed as limiting the invention.

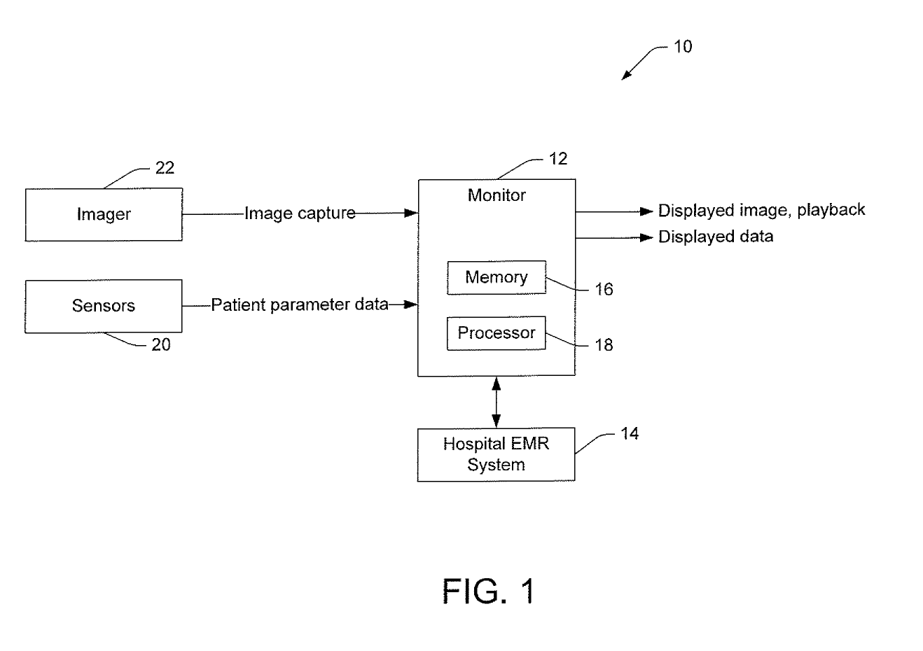

FIG. 1 illustrates a system for monitoring a patient to obtain objective data about the patient's condition, and to minimize subjectivity and documentation time related to other patient parameters that are typically difficult to quantify.

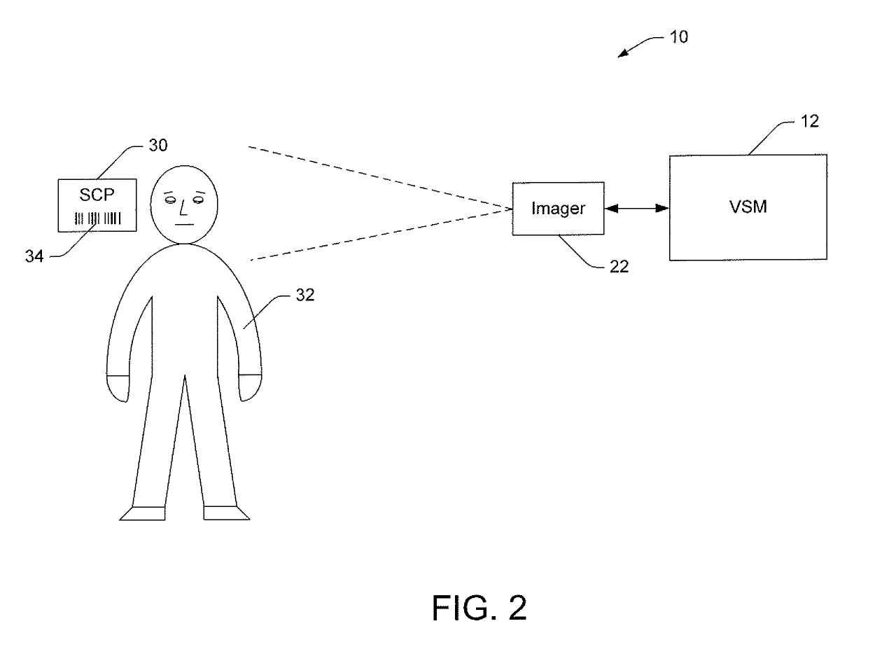

FIG. 2 illustrates another embodiment of the system, including a standard color plate (SCP) that facilitates calibrating an image of a patient in order to ensure that a displayed image correctly represents patient skin tone when reviewed by healthcare personnel who may be remote to the patient in location and/or time.

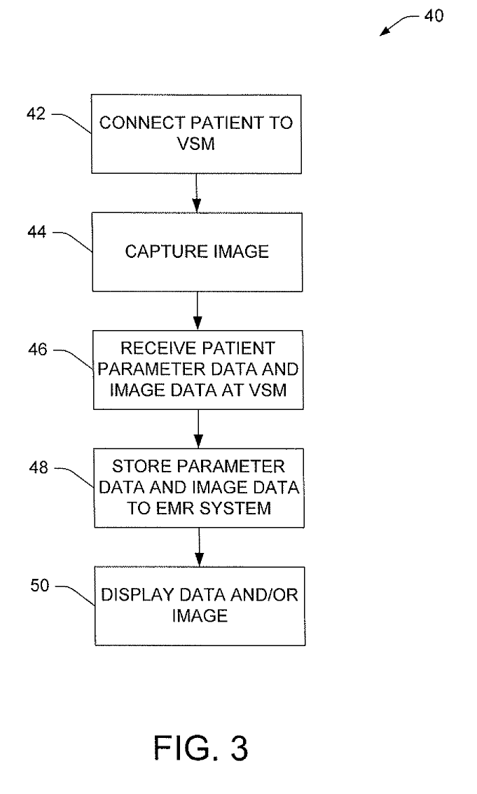

FIG. 3 illustrates a method for collecting image data in addition to vital sign information for a patient in a general healthcare environment.

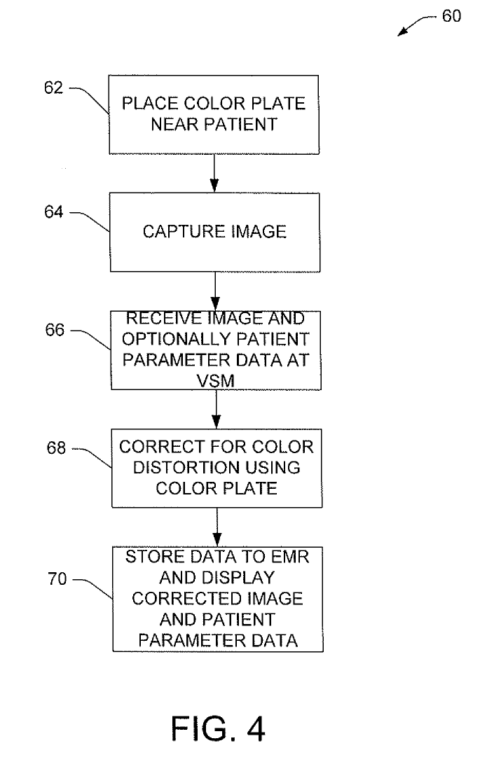

FIG. 4 is an illustration of a method for color correction of images generated using an imaging device coupled to a VSM, in conjunction with various aspects.

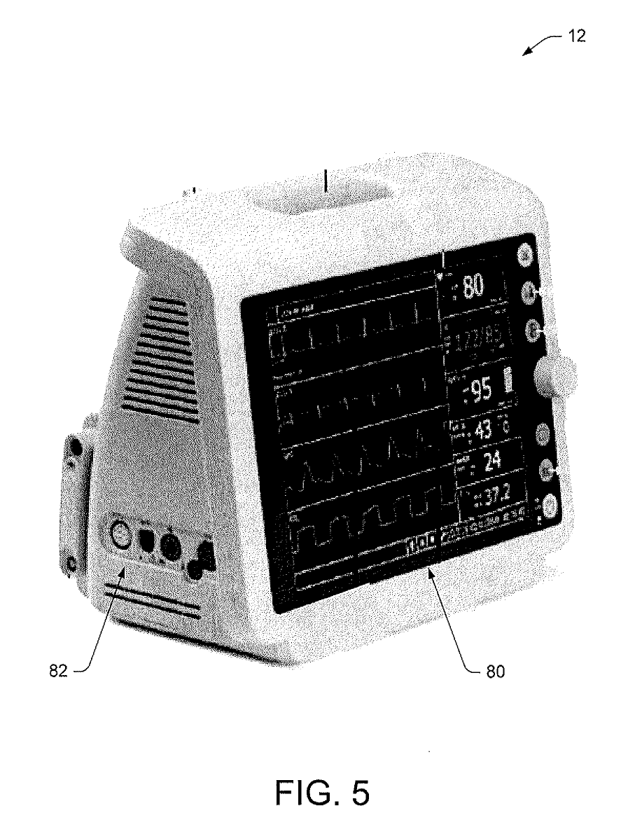

FIG. 5 is an illustration of a vital signs monitor, such as VSM, which can be employed in conjunction with the systems and/or methods described above.

FIG. 1 illustrates a system 10 for monitoring a patient to obtain objective data about the patient's condition, and to minimize subjectivity and documentation time related to other patient parameters that are typically difficult to quantify. The system includes a vital signs monitor (VSM) 12 that is connected to an electronic medical record (EMR) system 14. The connection between the VSM and the EMR can be wired or wireless (e.g., Bluetooth, Zigbee, etc.) as well as cellular. In some embodiments the connection is intermittent, and data transmission occurs whenever connectivity is sufficient, which may be all or a portion of the time. The VSM comprises a memory 16 and a processor 18. The memory stores information associated with patient parameter data (e.g., vital signs) received from one or more sensors 20 attached to the patient and/or image data captured by an imager 22. Patient parameter data can include any measurable patient parameter, such as heart rate or pulse, blood pressure, etCO₂, spO₂, temperature, blood-glucose levels, EKG, etc. The memory additionally stores one or more computer-executable routines or algorithms for receiving, processing, improving, storing, transmitting and/or displaying patient parameter data and/or image data. In one embodiment, the imager 22 is a barcode scanner or reader, which is capable of capturing 2D images. In another embodiment, the imager is a digital camera or video recorder.

For example, some bedside monitors, or VSMs, currently have a handheld two-dimensional barcode reader connected by a cable as a peripheral device. The barcode reader is used for reading barcodes on patient ID bracelets, pharmaceutical bottles, and the like. One of the features the 2D barcode reader is that the reader can also be used as an image input device. The barcode reader can thus be used as an image input device for inputting medically-related images, such as images of a wound to monitor its healing progress, dressings, particularly for bleed-through, punctures at catheter entry points and the like, and the patient's face to check for pallor, flushed appearance, bruising, etc. For example, contusion surface area size may be imaged, and contusion depth can be estimated from the color of the contusion, which in turn permits a physician to hypothesis on the force or source of the impact that caused the contusion, the blood volume lost to the interstitial space beneath the skin, etc. In another embodiment, rather than using the two-dimensional barcode reader, a digital camera can be associated with each VSM as the imager 22. Alternately, an attending medic, nurse, or physician can carry a camera device that interfaces with each of a plurality of VSMs. These images are then made part of the patient record analogous to the monitored vital signs.

The system 10 may be used in a general care medical field. The systems can also be used in trauma, battlefield, EMS, and similar monitors. Sending image(s) along with vital sign information ahead to the surgeons can facilitate better preparing the hospital staff for the trauma that they will soon need to treat. Various aspects of the invention include the ability to capture an image of a patient condition, to send images to a hospital EMR system or database, and to display an image on the VSM. According to other aspects, the imager can be embedded in the monitor or used in combination with the monitor as an accessory (e.g., such as the barcode reader or digital camera described above). Additionally, the system 10 can be used with adult, pediatric and/or neonatal patients in hospitals and/or out-of-hospital patient care settings (e.g., clinics, out-patient surgery facilities, long-term care facilities, physician offices, first-response sites and/or ambulances or other patient transporters, battlefield sites or temporary hospital sites. Having the ability to capture an image of the patient's condition using the vital signs monitor will shorten both the clinician's input time and the physician's review time.

According to other embodiments, the system 10 can be employed for training purposes, such as for medics, paramedics, emergency medical technicians, etc. For instance, a medic can take images of patients during a training (or real) triage exercise, and can later be critiqued by a superior such as a nurse or physician. In another example, a user can image a patient's facial expression and/or color (e.g., pallor, flush, etc.) to facilitate justifying a particular diagnosis in a training exercise. Still other image subjects can include wounds, dressings, catheter insertion points, etc. For instance, a wound on a patient can be imaged on daily schedule to permit a physician to evaluate healing progress, infection or the like.

FIG. 2 illustrates another embodiment of the system 10, including a standard color plate (SCP) 30 that facilitates calibrating an image of a patient in order to ensure that a displayed image correctly represents patient skin tone when reviewed by healthcare personnel who may be remote to the patient in location and/or time. The system 10 includes the VSM 12 and imager 14, which may be a digital camera, a video camera, a barcode reader, etc. The system further includes the SCP 30, which is placed proximate to the patient 32 or body part to be imaged before the image is generated. Additionally, the SCP includes a barcode 34 that is read by the imager, when the imager is a barcode reader at or near the time of generation of the image. In this manner, color calibration of the image input device can be performed. For measuring pallor, flush, or other aspects of patient skin coloration, one or more SCPs can be imaged concurrently with the patient or immediately before or after. Due to the wide range of skin tones with patients of different ethnicity, there may be several different color plates, each of which is identified by a barcode, and each of which corresponds to one or more different skin tones. It will be noted that the image color will be affected by lighting in the room, sunlight, etc., which may vary with the time of day, lighting level, and the like.

For instance, the barcode can represent information that describes the color(s) displayed on the SCP used in the image. According to an example, if the SCP is red, the barcode contains information that indicates that SCP, and may additionally contain information related to the particular hue, brightness, and/or shade of the indicated color(s). In another example, the SCP comprises multiple colors (e.g., red, blue, green, etc.) that are employed to calibrate the image to compensate for distortion that can be caused by lighting, shadows. A plurality of known colors can be used to define a custom transform for each image to map the colors in the electronic image to a pre-selected standardized color pallet. In this example, the VSM and/or a computer displaying the image to a physician can adjust color in the image in accordance with known values for the colors indicated in the barcode, thereby compensating for lighting and other distortional effects and ensuring that the physician is presented with a true-color image for diagnoses or evaluation.

Yet another example relates to a plurality of skin-toned SCPs that can be placed near the patient's face before imaging to facilitate determining whether the patient is pale, flushed, or the like. For example, pallor can indicate blood loss, other illness, etc. A flushed face can be indicative of fever, hyperglycemia (e.g., distinguishable in conjunction with certain other symptoms/signs, such as delirium, slow onset, etc.), and the like. Moreover, a bluish color in the patient's lips can be indicative of hypoxia, hypothermia, etc. The foregoing represent but a few examples of the types of information that can be quickly and efficiently captured by an imaging component using the system 10, without requiring laborious and time-consuming manual entry be a clinician and redundant interpretation by a physician. As will be appreciated, these and myriad other conditions can be documented, and image information can be color-compensated to ensure that a reviewing physician is presented with reliable and accurate image data without the delay and potential for errors and omissions that can occur when relying on a clinician to accurately document these relatively subjective patient symptoms and/or signs.

In an embodiment wherein the imager 22 includes a barcode reader, the reader can be a pen-type barcode reader, a laser scanner, a charge-coupled device (CCD) reader or LED scanner, etc. In such cases, imaging capability is often somewhat limited, and a digital camera may be included in the imager 22 to generate the images, while the barcode reader is employed to scan the barcode on the SCP. In other embodiments, the imager is a 2D imaging scanner or camera-based reader, which employs a small video camera to capture an image of the barcode 34. The camera can thus also be used to capture an image of the patient or body part. Such readers typically employ a digital image processing algorithm to decode the bar code. The video camera can employ CCD-like technology, but using multiple rows of sensors that facilitate 2D image generation.

In embodiments wherein the imager 22 is a digital camera or the like, the camera can be used to capture images that can be uploaded to the VSM upon capture, such as by a USB or similar connection, a wireless connection (e.g., Bluetooth, Zigbee, etc.), or the like. Alternatively, the camera can store the pictures for later uploading to the VSM and eventual storage to the EMR and presentation to a physician. Storage of image and vital sign information to the EMR can similarly be performed continuously as the information is processed by the VSM, or can be performed periodically, when a connection is provided between the VSM and the EMR database, or both.

According to an example relating to battlefield medicine applications, a medic can connect the VSM to a patient, obtain patient parameter information (vital signs), image the patient, wound dressing, etc., using the 2D barcode reader, and store the image and vital sign information to the VSM with some indicia of the patient identity to which the information corresponds. In some embodiments, a patient ID is associated with a barcode and/or patient ID number that is assigned to the patient and optionally printed on a patient wristband that is worn by the patient. The medic then moves on to a second patient and repeats the procedure. This process may be iterated indefinitely until the memory in the VSM is full. According to one embodiment, image resolution is selectable, and may be adjusted when storage space is limited. The medic can then send the VSM with the patients to a hospital or the like, where the VSM is connected to a hospital computer or work station (e.g., via a cabled or wireless connection), and the image and vital sign data is downloaded to respective patients' EMRs. The image and vital sign data can be viewed on the VSM (or on a workstation monitor) by a physician to assess patient condition. According to a related example, image and vital sign data can be forwarded to the hospital using a wired or wireless connection to an Ethernet portal, a cellular communication protocol, or some other technique, in order to give advance information to hospital staff regarding one or more incoming patients and their respective conditions.

According to another example, a clinician at a nursing home can attach the VSM to an elderly patient during a daily or weekly examination, and can image the patient to generate a record of patient condition for review by a remotely located physician. For instance, the patient may have a catheter entry point that has become infected, and treatment has been initiated. VSM collects vital sign information, and the clinician can capture an image of the catheter entry point. The collected and captured data is then stored to the VSM, and optionally to the patient's EMR. Access to the EMR database can be gained through an Ethernet connection or modem coupled to the VSM. Additionally, or alternatively, the VSM is coupled to a workstation (e.g., by cable or wireless connection) at the nursing home, which in turn provides the Ethernet connection for updating the EMR database. The remotely located physician can then inspect the vital sign information and image(s) to evaluate patient conditions (e.g., inflammation at the catheter site, rash, other visible infection, etc.) without having to make a house call to the patient and without having to wait for and/or rely upon the clinician's manually-generated description of the infected area.

FIGS. 3-4 illustrate one or more methods related to recording images related to patient condition, in addition to monitored patient parameter (e.g., vital sign) information, to mitigate delay and error that can occur using conventional manual entry methods, in accordance with various features. While the methods are described as a series of acts, it will be understood that not all acts may be required to achieve the described goals and/or outcomes, and that some acts may, in accordance with certain aspects, be performed in an order different that the specific orders described.

FIG. 3 illustrates a method 40 for collecting image data in addition to vital sign information for a patient in a general healthcare environment. At 42, a patient is connected to a VSM. Connecting the patient to the VSM can include positioning one or more sensors on or about the patient. At 44, an image of the patient or a portion of the patient is captured. For instance, a clinician can employ a 2D barcode scanner or digital camera coupled to the VSM to capture a picture of the patient. From the picture, a condition of the patient is evaluated, such as whether the patient is flushed, pale, in discomfort, etc., and the source of the evaluated condition can be more accurately predicted.

At 46, image data and patient parameter data are received at the VSM. The VSM can store to an EMR for the patient at 48. The EMR can be maintained in the VSM and/or in an EMR database remote from the VSM. At 50, image data and/or a snapshot of the patient's monitored vital sign data is displayed. Display of the data can be executed on the VSM, such as on a VSM screen, or can be presented on a workstation or computer coupled to the VSM or that has received the data from the VSM. In this manner, a physician can review image data associated with the patient, a wound, a wound dressing, etc., as well as data related to the patient's vital signs at or about the time of the image.

FIG. 4 is an illustration of a method 60 for color correction of images generated using an imaging device coupled to a VSM, in conjunction with various aspects. At 62, a standard color plate is positioned near a patient to be imaged. For instance, the SCP can be placed next to the patient's head, on the patient's chest, or otherwise in the frame of the picture or image to be generated. According to some aspects, the SCP has a barcode printed on it, which contains information related to the specific color, hue, brightness, shade, contrast, etc., printed on the SCP. The barcode is scanned at or about the time of the image capture to permit the VSM to adjust for lighting conditions and the like, based on the colors received in the image data as compared to expected colors based on the barcoded color identification information. In some embodiments, the SCP contains the barcode and/or at least one reference color.

At 64, the image is captured. If the imaging device is a barcode reader, the image is captured by depressing a trigger or other activation mechanism while the device is aimed at the target. In other embodiments, the device is a digital camera that is operated by depressing a button or the like to take a picture. At 66, the VSM receives the image data from the imaging device, as well as patient vital sign data (e.g., from sensors attached to the patient.

At 68, the VSM corrects for color distortion using the information contained in the SCP. For instance, if the barcode on the SCP indicates that the SCP is a bright red color (e.g., for calibration purposes), but the color plate appears dingy red in the image, then it may be assumed that the lighting in the area in which the image was generated is insufficient. Accordingly, the VSM can brighten the image until the color plate exhibits a color, hue, shade, etc., that approximates its true color and/or expected color based on the barcode information. It will be understood that the VSM includes sufficient memory and processing capability to store and execute one or more color compensation algorithms. In one embodiment, the SCP has a plurality of predefined color samples that are mapped to a standardized pallet. The diagnostic portion of the image is mapped analogously such that the image is transformed to a standardized color pallet. In other embodiments, the SCP is provided in the image for comparison by a clinician, physician, or other user, such that the user can see that the SCP is not as bright as expected and can infer that all other colors in the image are similarly dimmed or shaded. In this manner, the user can perform color compensation intuitively.

At 70, the data (e.g., image data and patient vital sign data) is stored to the patient's EMR, and the color-corrected image and vital sign data are displayed to a user for review. In this manner, patient information that is not measurable by the VSM (e.g., pallor, skin tone, rash, etc.) is documented, stored, and reviewed without requiring a clinician to handwrite, type, or otherwise enter the information. By providing images to the physician, the physician's opinion and diagnosis are shielded from bias that can occur when reviewing a clinician's observations second hand.

FIG. 5 is an illustration of a vital signs monitor, such as VSM 12, which can be employed in conjunction with the systems and/or methods described above. The monitor 12 comprises a screen 80 that displays information to a user. For instance, the screen can display vital sign information related to a patient to whom the monitor 12 is connected. The monitor 12 also has a plurality of connection ports 82, such as a PS/2 port, a USB port, and the like, which are employed by a user to connect the barcode reader, digital camera, sensor leads, etc. It will be appreciated that the monitor 12 can monitor any and all suitable or desired patient-related conditions, including but not limited to blood pressure, temperature, heart rate, SpO₂, exhaled CO₂, blood-glucose levels, electrocardiogram (ECG/EKG) related information, etc.

The monitor additionally can be provided with software that executes instructions for providing the functionality of the systems and/or methods described above. For instance, color correction software can be provided to the VSM using a USB stick or the like. Additionally, imaging software can be stored and/or executed in the VSM, to permit images captured by a scanner or camera to be presented to a user on the screen 80.

1. A system for concurrently capturing vital sign data and an image of a patient, including:
a vital signs monitor that receives patient parameter data and image data;
an imager, coupled to the VSM, that captures an image of a patient and transmits image data to the VSM; and
one or more sensors that monitor at least one patient vital sign and transmit patient parameter data describing the at least one vital sign to the VSM.

2. The system according to claim 1, further including a standard color plate (SCP) that is associated with the image of the patient.

3. The system according to claim 2, wherein the SCP has at least one portion colored to approximate the normal skin tone of the patient.

4. The system according to claim 2, further including a barcode positioned on the SCP, the barcode includes information related to the color of the SCP.

5. The system according to claim 4, wherein the imager includes a barcode reader that captures the image of the patient, or a portion thereof, and transmits the image data to the VSM for storage in an electronic medical record (EMR) or display to a physician.

6. The system according to claim 5, wherein the barcode reader reads the barcode, and wherein the VSM employs information obtained from the barcode to calibrate at least one of color, contrast, or brightness in the image.

7. The system according to claim 1, wherein the imager includes a barcode reader that captures the image of the patient, or a portion thereof, and transmits the image data to the VSM for storage in an electronic medical record (EMR) or display to a physician.

8. The system according to claim 1, wherein the image of the patient is an image of at least one of the patient's face, a wound on the patient, a wound dressing on the patient, or a catheter insertion point.

9. The system according to claim 8, wherein the image of the patient's face is evaluated for information related to at least one of skin tone or expression of pain, for diagnosis of the patient's condition.

10. The system according to claim 1, wherein the imager is a digital camera that captures images of the patient, or a portion thereof, and transmits the image data to the VSM for storage to an EMR or presentation to a physician, or both.

11. The system according to claim 2, wherein the memory includes:
a routine or means for capturing the image of the patient and the SCR;
a routine or means for receiving the image data and optionally receiving patient parameter data at the VSM;
a routine or means for correcting for color distortion in the image at the VSM; and
a routine or means for storing the image and optional patient parameter data at the VSM and displaying the stored data to a user.

12. A method of capturing vital sign and image data using the system of claim 1, including:
connecting the patient to the VSM using the one or more sensors;
capturing an image of the patient using the imager;
receiving the image data and patient parameter data at the VSM;
storing the image data and patient parameter data to an EMR database; and
displaying the image and patient parameter data to a user.

13. A method of concurrently capturing vital sign information and an image of a patient, including:
connecting one or more sensors to a patient and to a vital signs monitor (VSM);
receiving patient parameter data descriptive of the patient's vital signs at the VSM;
capturing an image of the patient;
receiving the captured image at the VSM;
storing the captured image and the patient parameter data to an electronic medical record (EMR); and
displaying the patient parameter data and the captured image.

14. The method according to claim 13, further including employing a digital camera, coupled to the VSM, to capture the image.

15. The method according to claim 13, further including employing a two-dimensional barcode reader, coupled to the VSM, to capture the image.

16. The method according to claim 15, further including using the barcode reader to read a patient-identifying barcode.

17. The method according to claim 15, further including positioning a standard color plate (SCP) on or near the patient, and capturing the image of the patient with the SCP in the image.

18. The method according to claim 17, further including employing the imaged SCP for color-calibration of the captured image.

19. The method according to claim 15, further including, with the barcode reader, reading a barcode associated with the SCP, the barcode including information related to the color of the SCP.

20. The method according to claim 19, further including scanning the barcode at or near the time of capturing the image, and employing the barcode information in correcting for color distortion in the captured image.

21. The method of claim 20, further including displaying the captured image and the patient parameter data to a user on at least one of the VSM and a workstation monitor, wherein the workstation has access to a database storing the EMR.

22. A processor or computer-readable memory programmed to perform the method of claim 13.

23. A system that facilitates configuring multiple patient monitoring devices, including:
means for monitoring patient vital signs;
means for capturing an image of a monitored patient;
means for storing patient vital sign information and a captured image of the patient as an electronic medical record; and
means for providing a standard reference for color correction of the captured image.
